(19) United States
(12) Patent Application Publication
(10) Pub. No.: US 2002/0037256 A1
(43) Pub. Date: Mar. 28, 2002

(54) Composition packaged in an aerosol device, comprising alumina nanoparticles

(76) Inventors: Cecile Nocerino, Paris (FR); Franck Giroud, Clichy (FR); Jean-Michel Sturla, Boulogne (FR)

Correspondence Address: STEPTOE & JOHNSON LLP, 1330 Connecticut Ave., N.W., Washington, DC, 20036, US

(21) Appl. No.: 09/955,299

(22) Filed: Sep. 19, 2001

(30) Foreign Application Priority Data
Sep. 20, 2000 (FR) 0011991

(51) Int. Cl. A61K 7/00; A61K 9/14

(52) U.S. Cl. 424/46; 424/47

(57) ABSTRACT

The present invention relates to a composition packaged in an aerosol device comprising a liquid phase and a propellant. The liquid phase comprises, in a cosmetically acceptable medium, nanoparticles comprising at least 10% by weight of alumina and exhibiting a number-average primary size of between 2 and 200 nm. The propellant is chosen from dimethyl ether, C₃₋₅ alkanes, 1,1-difluoroethane, mixtures of dimethyl ether and of C₃₋₅ alkanes or mixtures of 1,1-difluoroethane and of dimethyl ether and/or of C₃₋₅ alkanes. The invention also relates to a process for the cosmetic treatment of the hair and to the use as styling product.

The present invention relates to a composition packaged in an aerosol device comprising a propellant and a liquid phase which comprises, in a cosmetically acceptable medium, alumina nanoparticles, to a process for the cosmetic treatment of the hair and to the use as styling product.

Products such as, for example, lacquers, foams and gels are well known in the art for styling the hair by fixing the individual hairs to one another.

However, simply passing the fingers, a comb or a brush through leads to breaking of the welds created during the application of these conventional products. They therefore do not make possible restyling of the hair.

U.S. Pat. No. 3,819,827 from Wella discloses in particular products for the setting of hair comprising from 0.2 to 6% by weight of aluminium oxide particles exhibiting a particle size of approximately 30 _m.

The Applicant Company has found, surprisingly and unexpectedly, that the use in an aerosol of nanoparticles comprising at least 10% by weight of alumina and exhibiting a number-average primary size of between 2 and 200 nm in a cosmetically acceptable medium makes it possible to give volume to the hair and to obtain styling without fixing and overloading the hair. The hair can then be styled and restyled at will while a retaining a natural appearance.

The term <<cosmetically acceptable medium>> is understood to mean a medium which is compatible with the hair but which also has a pleasant smell, appearance and feel.

A subject-matter of the invention is thus a composition, packaged in an aerosol device, comprising a specific propellant and a liquid phase which comprises, in a cosmetically acceptable medium, nanoparticles comprising at least 10% by weight of alumina and exhibiting a number-average primary size of between 2 and 200 nm.

Another subject-matter of the present invention is a process for the cosmetic treatment of the hair employing the composition according to the invention.

A further subject-matter of the invention is the use of the composition according to the invention as styling product.

Other subject-matters, characteristics, aspects and advantages of the invention will become even more clearly apparent on reading the description and various examples which follow.

According to the invention, the cosmetic composition packaged in an aerosol device comprises a propellant, chosen from dimethyl ether, C₃₋₅ alkanes, 1,1-difluoroethane, mixtures of dimethyl ether and of C₃₋₅ alkanes or mixtures of 1,1-difluoroethane and of dimethyl ether and/or of C₃₋₅ alkanes, and a liquid phase which comprises, in a cosmetically acceptable medium, nanoparticles comprising at least 10% by weight of alumina and exhibiting a number-average primary size of between 2 and 200 nm and preferably between 5 and 50 nm.

The particles according to the invention can, for example, have any shape, for example the shape of spheres, flakes, needles or slabs, or completely random shapes.

Within the meaning of the present invention, the term <<primary particle size>> is understood to mean the maximum dimension which it is possible to measure between two diametrically opposite points of an individual particle. The size can be determined by transmission electron microscopy or from the measurement of the specific surface area by the BET method.

In accordance with the present invention, the alumina-comprising solid particles form a mass in which the alumina does not act as coating agent for one (or other) filler(s).

In the case where the nanoparticles are formed of alumina and other fillers, the alumina is found in the free state and does not form chemical bonds with other fillers. There is then present a blend between the alumina and other fillers, in particular with metal or semimetal oxides, in particular obtained by thermal fusion of these various constituents.

When the nanoparticles comprising at least 10% by weight of alumina additionally comprise a metal or semimetal oxide other than alumina, the latter is chosen in particular from silicon oxide or boron oxide.

Preferably, the nanoparticles comprise at least 50% by weight of alumina, better still at least 70% by weight, and the nanoparticles formed of more than 90% by weight of alumina are particularly preferred according to the present invention.

The alumina which is suitable in the compositions of the present invention is preferably an optionally hydrated alumina, such as, for example, boehmite.

The alumina-comprising nanoparticles according to the invention are used in particular in an amount of between 0.01 and 30% by weight and preferably between 0.05 and 5% by weight with respect to the total weight of the composition.

The liquid phase can also comprise other types of nanoparticles, for example titanium oxide or zinc oxide nanoparticles.

The cosmetically acceptable medium comprises water and/or a cosmetically acceptable solvent chosen in particular from lower C₁-C₄ alcohols, such as ethanol, isopropanol, tert-butanol and n-butanol; polyols, such as propylene glycol; polyol ethers; acetone; and their mixtures. The solvent which is particularly preferred in the invention is ethanol.

This cosmetically acceptable medium preferably comprises an amount of water of less than 20% by weight of the composition.

The liquid phase of the composition according to the invention can additionally comprise conventional cosmetic additives chosen from adhesive agents, reducing agents, such as thiols, fatty substances, thickening agents, softeners, antifoaming agents, screening agents, antiperspirants, acidifying agents, basifying agents, dyes, pigments, fragrances, preservatives, surfactants, fixing or nonfixing polymers, volatile or nonvolatile silicones, vegetable, animal, mineral or synthetic oils, proteins and vitamins, and their mixtures.

Mention may be made, as particularly preferred additive, of, for example, vinyllactam homopolymers or copolymers well known in the art.

A person skilled in the art will take care to choose the possible additives and their amount so that they do not harm the properties of the compositions of the present invention.

These additives are in particular present in the composition according to the invention in an amount ranging from 0 to 20% by weight with respect to the total weight of the composition.

The propellant is in particular present in an amount of between 2 and 90% by weight, preferably between 40 and 90%, better still between 40 and 80% by weight, with respect to the total weight of the composition.

Preferably, the aerosol device according to the invention comprises, as propellant, dimethyl ether, alone or as a mixture.

The compositions in accordance with the invention are packaged in an aerosol device conventional in cosmetics.

The sprayed compositions can be provided in the form of a spray or of a foam.

The compositions in accordance with the invention, sprayed from the aerosol device, can be used in a rinse-out or leave-in application, as compositions for fixing and/or retaining the form of the hair, hair care compositions, shampoos, compositions for conditioning the hair, such as compositions intended to impart softness on the hair, or compositions for making up the hair.

The present invention also relates to a process for the cosmetic treatment of the hair which consists in applying an effective amount of a composition as described above to the hair and in rinsing out or not rinsing out, after an optional setting time.

According to a preferred embodiment of the invention, the composition sprayed from the aerosol device can be used as leave-in styling product.

The following example illustrates the present invention and should not be regarded in any way as limiting the invention.

### EXAMPLE

A styling product was prepared from the following ingredients, the percentages being expressed by weight.

Aluminium oxide nanopigment⁽¹⁾ 0.3%

Water 2.7%

Ethyl alcohol 32%

Dimethyl ether 65%

⁽¹⁾ exhibiting a number-average primary particle size of 13 nm, sold under the name Aluminium Oxide C by Degussa-Hüls.

The ingredients are mixed in a can equipped with a valve and an atomizer. This composition is sprayed over the hair and is left to dry for a few seconds.

The hair has a large volume. It is sleek and easy to style and to restyle.

1. Composition packaged in an aerosol device comprising a liquid phase, which comprises, in a cosmetically acceptable medium, nanoparticles comprising at least 10% by weight of alumina and exhibiting a number-average primary size of between 2 and 200 nm, and a propellant chosen from dimethyl ether, C₃₋₅ alkanes, 1,1-difluoroethane, mixtures of dimethyl ether and of C₃₋₅ alkanes or mixtures of 1,1-difluoroethane and of dimethyl ether and/or of C₃₋₅ alkanes.

2. Composition according to claim 1, characterized in that the number-average primary size of the nanoparticles is between 5 and 50 nm.

3. Composition according to claim 1 or 2, characterized in that the nanoparticles comprising at least 10% by weight of alumina additionally comprise a metal or semimetal oxide other than alumina.

4. Composition according to claim 3, characterized in that the metal or semimetal oxide is chosen from silicon oxide or boron oxide.

5. Composition according to any one of the preceding claims, characterized in that the nanoparticles comprise at least 50% by weight of alumina.

6. Composition according to any one of the preceding claims, characterized in that the nanoparticles are formed of more than 90% by weight of alumina.

7. Composition according to any one of the preceding claims, characterized in that the alumina is an optionally hydrated alumina.

8. Composition according to claim 7, characterized in that the alumina is boehmite.

9. Composition according to any one of the preceding claims, characterized in that the amount of the nanoparticles is between 0.01 and 30% by weight, preferably between 0.05 and 5% by weight, with respect to the total weight of the composition.

10. Composition according to any one of the preceding claims, characterized in that propellant is composed of dimethyl ether, alone or as a mixture.

11. Composition according to any one of the preceding claims, characterized in that the propellant is present in an amount of between 2 and 90% by weight, preferably between 40 and 80% by weight, with respect to the total weight of the composition.

12. Composition according to any one of the preceding claims, characterized in that the cosmetically acceptable medium comprises water and/or a cosmetically acceptable solvent.

13. Composition according to claim 12, characterized in that the solvent is chosen from lower C₁-C₄ alcohols, polyols, polyol ethers, acetone, and their mixtures.

14. Composition according to claim 13, characterized in that the solvent is ethanol.

15. Composition according to claim 12, characterized in that the water represents less than 20% by weight of the composition.

16. Composition according to any one of the preceding claims, characterized in that the liquid phase additionally comprises conventional cosmetic additives chosen from adhesive agents, reducing agents, such as thiols, fatty substances, thickening agents, softeners, antifoaming agents, screening agents, antiperspirants, acidifying agents, basifying agents, dyes, pigments, fragrances, preservatives, surfactants, fixing or nonfixing polymers, volatile or nonvolatile silicones, vegetable, animal or mineral oils, proteins and vitamins, and their mixtures.

17. Composition according to claim 16, characterized in that it comprises a vinyllactam homopolymer or copolymer.

18. Process for the cosmetic treatment of the hair, characterized in that the composition according to any one of the preceding claims is applied to the hair and in that rinsing is or is not carried out, after an optional setting time.

19. Use of the composition according to any one of claims 1 to 17, sprayed from the aerosol device, as styling product, preferably leave-in styling product.
